(19) United States
(12) Patent Application Publication
(10) Pub. No.: US 2004/0204390 A1
(43) Pub. Date: Oct. 14, 2004

(54) Pharmaceutical combination comprising an anti-androgen and an oestrogen receptor beta agonist

(76) Inventors: Barrington John Albert Furr, Cheshire (GB); Anthony Nigel Brooks, Cheshire (GB); Chris Allan Veale, Wilmington, DE (US)

Correspondence Address: Ropes & Gray, One International Place, Boston, MA, 02110-2624, US

(21) Appl. No.: 10/477,242

(22) PCT Filed: May 8, 2002
(86) PCT No.: PCT/GB02/02125

(30) Foreign Application Priority Data
May 14, 2001 (SE) 0101697-1

(51) Int. Cl. A61K 31/56

(52) U.S. Cl. 514/171

(57) ABSTRACT

The present invention relates to a pharmaceutical product or daily dose comprising an anti-androgen, an oestrogen receptor β (ERβ) selective agonist and optionally a chemical castration agent. The invention also relates to a method of therapeutically treating and/or preventing an androgen-stimulated disease (eg, prostate cancer or benign prostate hypertrophy) in a patient. Furthermore, the invention relates to the use of an anti-androgen, an ERβ selective agonist and optionally a chemical castration agent in the manufacture of a pharmaceutical product for this purpose.

### CROSS-REFERENCE TO RELATED APPLICATIONS

This application is a national stage filing under 35 U.S.C. 371 of International Application No. PCT/GB02/02125, filed May 8, 2002, which claims priority from Sweeden Application No. 0101697.1, filed May 14, 2001, the specification of which is incorporated by reference herein. International Application No. PCT/GB02/02125 was published under PCT Article 21(2) in English.

### FIELD OF THE INVENTION

The present invention relates to a pharmaceutical product or daily dose comprising an anti-androgen, an oestrogen receptor β (ERβ) selective agonist and optionally a chemical castration agent. The invention also relates to a method of therapeutically treating and/or preventing an androgen related condition (eg, prostate cancer or benign prostate hypertrophy) in a patient. Furthermore, the invention relates to the use of an anti-androgen, an ERβ selective agonist and optionally a chemical castration agent in the manufacture of a pharmaceutical product for this purpose.

### BACKGROUND OF THE INVENTION

Prostate glands, in particular the epithelial cells thereof, both malignant and benign, are generally hormone-dependent and, thereby, sensitive to inhibition of androgen-driven growth signalling by way of the androgen receptor. Androgen ablation may be achieved by way of surgical castration or chemical castration, for example using a luteinising hormone releasing hormone (LHRH) agonist such as goserelin or leuprorelin or a LHRH antagonist. The effects of androgens may also be countered using anti-androgen therapy, for example, using an anti-androgen such as bicalutamide (or an enantiomer thereof), flutamide or nilutamide, which act at the androgen receptor. The properties and usefulness of these anti-androgens have been reviewed, for example in the following documents which are incorporated herein by way of reference:

Flutamide R O Neri, J. Drug Develop., 1987, 1 (Suppl.), 5-9 and Urology, 1989, 34 (Suppl. 4), 19-21 and United Kingdom Patent Application No. 1360001;

bicalutamide B J A Furr et al., Urology, 1996, 47 (Suppl. 1A), 13-25, G J C Kolvenbag et al., Urology, 1996, 47 (Suppl. 1A), 70-79 and European Patent Application No. 0100172 as the 8th compound listed in the table in Example 6;

nilutamide M G Harris et al., Drugs and Aging, 1993 3, 9-25 and United Kingdom Patent Application No.1518444.

Bicalutamide, a non-steroidal anti-androgen, is the racemate of 4′-cyano-α′,α′,α′-trifluoro-3-(4-fluorophenylsulphonyl)-2-hydroxy-2-methylpropiono-m-toluidide and is known by the AstraZeneca trade name CASODEX™. EP-100172 discloses 4′-cyano-α′,α′,α′-trifluoro-3-(4-fluorophenylsulphonyl)-2-hydroxy-2-methylpropiono-m-toluidide (named in EP-100172 as 4-cyano-3-trifluoromethyl-N-(3-p-fluorophenylsulphonyl-2-hydroxy-2-methylpropionyl)aniline) as the 8^{th}compound listed in the table in Example 6. The corresponding structure is shown in formula I:

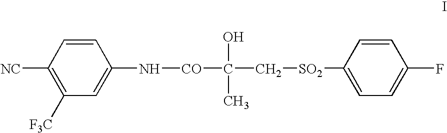

4′-cyano-α′,α′,α′-trifluoro-3-(4-fluorophenylsulphonyl)-2-hydroxy-2-methylpropiono-m-toluidide can exist in distinct R— and S-enantiomeric forms. The R-enantiomer is the (−) isomer and is the pharmacologically active compound in vivo. For further details of the enantiomers, reference is made to Tucker and Chesterton, J. Med. Chem. 31, pp 885-887 (1988).

Flutamide, an anti-androgen, is known by the trade name EULEXIN™. Flutamide is also known by the alternative names 2-methyl-N-[4-nitro-3-(trifluoromethyl)phenyl]propanamide; α, α, α-trifluoro-2-methyl-4′-nitro-m-propionotoluidide; and 4′-nitro-3′-trifluoromethylisobutyranilide. Flutamide is disclosed in U.S. Pat. No. 3,847,988. The corresponding structure is shown in formula II:

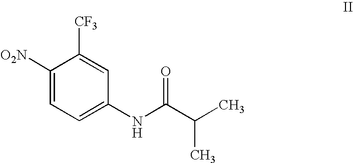

Nilutamide, an anti-androgen, is known by the trade name NILANDRON™. Nilutamide is also known by the alternative names 5,5-dimethyl-3-[4-nitro-3-(trifluoromethyl)phenyl]-2,4-imidazolidinedione; and 1-(3′-trifluoromethyl-4′-nitrophenyl)-4,4-dimethylimidazoline-2,5-dione. Nilutamide is disclosed in U.S. Pat. No. 4,097,578. The corresponding structure is shown in formula III:

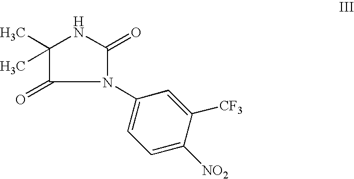

Chlormadinone, eg in its acetate form, is an anti-androgen. The acetate form is known by the alternative names 17-(acetyloxy)-6-chloropregna-4,6-diene-3,20-dione; 6-chloro-17-hydroxypregna-4,6-diene-3,20-dione acetate; 6-chloro-6-dehydro-17α-hydroxyprogesterone acetate; 6-chloro-6-dehydro-17α-acetoxyprogesterone; and 17α-acetoxy-6-choro-6,7-dehydroprogesterone. Chormadinone is disclosed in U.S. Pat. No. 3,485,852.

Cyproterone is known by the alternative names (1β,2β)-6-chloro-1,2-dihydro-17-hydroxy-3′H-cyclopropa[1,2]pregna-1,4,6-triene-3,20 dione; 6-chloro-17-hydroxy-1α,2α-methylenepregna-4,6-diene-3,20-dione; 6-chloro-6-dehydro-17α-hydroxy-1,2α-methyleneprogesterone; and 6-chloro-1,2α-methylene-4,6-pregnadien-17α-ol-3,20-dione. Cyproterone is disclosed in U.S. Pat. No. 3,234,093. Cyproterone acetate is an anti-androgen.

It would be desirable to counter the effects of androgens to a greater extent than is achieved by anti-androgen therapy alone. This would be useful in the treatment and/or prevention of androgen-related conditions, i.e. androgen-stimulated diseases, such as the prostate cancers discussed above, as well as conditions such as benign prostate hypertrophy (BPH).

The present invention achieves this by providing a pharmaceutical product comprising an anti-androgen, an oestrogen receptor β (ERβ) selective agonist and optionally a chemical castration agent, for simultaneous or sequential administration to a patient for therapeutically treating and/or preventing an androgen related condition in the patient.

The present invention also provides a daily pharmaceutical dose for administration to a patient for therapeutically treating and/or preventing an androgen-stimulated disease in the patient, the dose comprising an anti-androgen and an ERβ selective agonist, for simultaneous or sequential administration to the patient.

Another aspect of the invention relates to the use in the manufacture of a pharmaceutical product of an anti-androgen, an oestrogen receptor β (ERβ) selective agonist and optionally a chemical castration agent, for simultaneous or sequential administration to a patient, for therapeutically treating and/or preventing an androgen-stimulated disease in the patient.

A further aspect relates to method of therapeutically treating and/or preventing an androgen-stimulated disease in the patient comprising simultaneously or sequentially administering an anti-androgen, an oestrogen receptor β (ERβ) selective agonist and optionally a chemical castration agent to the patient.

For many years it was thought that only one oestrogen receptor (ERα) existed. More recently, a second oestrogen receptor (ERβ) has been discovered. The two receptors have different tissue distributions; ERα shows highest expression in uterus, testis, pituitary gland, ovary, kidney, epididymis and adrenal gland, whereas ERβ is expressed highest in brain, prostate, ovary, lung, bladder and epididymis. ERα and ERβ are encoded by different genes. The cloning of ERβ has been described in Proc. Natl. Acad. Sci. USA (1996), 93:5925-5930, Kuiper et al, “Cloning of a novel estrogen receptor expressed in rat prostate and ovary”. G Prins and L Birch have described the neonatal oestrogen treatment of male rats, which was shown to down-regulate androgen receptor expression in the epithelial cells of all three prostate lobes, thus leading to an overall prostate growth retardation (Endocrinology (1995), Vol. 136, No. 3, 1303-1314, G Prins and L Birch, “The development pattern of androgen receptor expression in rat prostate lobes is altered after neonatal exposure to estrogen”).

The present invention is based on the surprising synergistic effect produced by the combined use of an anti-androgen and an oestrogen receptor β (ERβ) selective agonist for therapeutically treating and/or preventing an androgen related condition (eg, early or advanced prostate cancer) in a patient. The combination can have a synergistic effect in terms of one or more of the extent of response, the response rate, the time to disease progression and the patient survival rate. It is expected that the combination will have a beneficial effect in preventing the onset of androgen-stimulated disease, eg, prostate cancer, in men genetically predisposed to the disease. Conventional methods are available to classify patients according to their risk of contracting cancer, for example by assessment of family history and measurements over time of particular blood proteins such as prostate specific antigen (PSA).

While not wishing to be bound by any particular theory, we believe that the synergistic effect is a result of the action of the anti-androgen at androgen receptors together with the action of the ERβ selective agonist causing down-regulation of androgen receptors in the patient. The invention calls for use of an agonist that is selective for ERβ because the inventors believe that the two estrogen receptors have differing and potentially opposite effects, whereby activation of ERα by an ERα selective agonist causes cellular proliferation and activation of ERβ by a selective ERβ agonist inhibits proliferation.

The term ‘androgen related condition’ is also referred to herein as ‘androgen-stimulated disease’ and refers to a disease that is activated or caused by androgen. Such diseases include benign and malignant prostate disease, acne and hirsutism.

The term “product” is intended to mean either a mixture of the anti-androgen and ERβ selective agonist (eg, provided as a capsule or tablet containing both compounds) or a kit comprising separate amounts of the compounds (eg, a set of ERβ selective agonist tablets and a separate set of tablets of the anti-androgen). The latter product can be used for simultaneous or sequential (ie, temporally spaced) administration of the compounds to the patient, while the pre-mixed compounds are for simultaneous administration. Factors such as the rate of absorption, metabolism and the rate of excretion of each agent will affect their presence at the tumour site. Such factors are routinely considered by, and are well within the ordinary skill of, the clinician when he contemplates the treatment of a medical condition which requires the conjoint administration of two agents in order to obtain a beneficial effect.

The patient can be a human male, eg an adult, but the treatment of other mammals is also contemplated.

In one embodiment of the present invention, the anti-androgen is selected from flutamide, nilutamide, bicalutamide or a pharmaceutically acceptable salt, enantiomer or solvate thereof, chlormadinone acetate and cyproterone acetate. For example, the anti-androgen can be the R-enantiomer of bicalutamide. Depending on the particular anti-androgen, suitable salts are, for example acid addition salts, such as hydrochloride, hydrobromide, phosphate, acetate, fumarate, maleate, tartarate, citrate, oxalate, methanesulphonate or p-toluenesulphonate, or alkali metal salts such as sodium or potassium salts.

Preferably, the ERβ selective agonist is selected from the group consisting of: genistein, diadzen and coumesterol, or an oestrogenic analogue thereof (such as those described in WO 00/62765) or a pharmaceutically acceptable salt, enantiomer or solvate ERβ selective agonist thereof. The term ERβ selective agonist means that the ERβ agonist is more selective for ERβ than ERα. In distinct embodiments, the agonist is, in increasing order of preference, 5-fold, 10-fold, 20-fold, 100-fold, or more, selective for ERβ than ERα. More preferably, the ERβ agonist is completely selective for ERβ, but not ERα. Agonists purported to be more selective for the ERβ are disclosed in WO 00/59897 and WO 00/62765, and the disclosure of these agonists including the disclosure of their manufacture is explicitly incorporated herein by reference in order to provide examples of agonists that may be used in the present invention. Further details on phytooestrogens are disclosed in Endocrinology 1998, v. 139, 10, pp4252-4263, which is explicitly incorporated herein by reference in order to provide examples of agonists that, if selective for ERβ, may be used in the present invention. The person skilled in the art is able to determine whether or not a test compound is a selective agonist for ERβ. A suitable estrogen receptor binding assay and a cell based ER transcriptional assay, to determine specificity, are described in the Examples section herein.

Where a chemical castration agent is used, this is for example selected from goserelin and leuprorelin.

In the pharmaceutical product, the anti-androgen and the ERβ selective agonist can, for example, be provided in a weight ratio of 25 to 1000 mg (preferably the lower end of the range being 50 or 100; preferably the upper end of the range being 500, 350, 300,1 50 or 50; suitable values in the ranges being 750, 375, 150, 125 or 50): 0.03 to 250 (preferably the lower end of the range being 0.1 preferably the upper end of the range being 10; the most preferred range being 0.3 to 2.5; a suitable value in the range being 0.3, 0.5, 0.75, 1, 1.25, 1.5, 2 or 2.5). For bicalutamide, a preferred range is 10 to 350 (preferably the lower end of the range being 50; preferably the upper end of the range being 300, 150 or 50; suitable values in the ranges being 150 or 50). For flutamide, a preferred range is 100 to 1000, and 750 or 375 is a preferred value. For chlormadinone acetate a preferred value is 50. For cyproterone acetate a preferred range is 200 to 300. For nilutamide, a preferred range is 50 to 500, and 300 or 150 is a preferred value.

In one embodiment, each compound of the pharmaceutical product of the invention is administered daily. Another possible regime would be dosing of the anti-androgen on alternate days and dosing of the ERβ selective agonist also on (the same or different) alternate days. Alternatively, the anti-androgen is administered every 3, 4, 5, 6, or 7 days and the ERβ selective agonist is administered every 3, 4, 5, 6, or 7 days (eg, on the same day as the anti-androgen). To this end, a kit may be provided comprising the pharmaceutical product together with dosing instructions.

In one example, the daily pharmaceutical dose comprises the anti-androgen in an amount of 25 to 1000 mg (preferably the lower end of the range being 50 or 100 mg; preferably the upper end of the range being 500, 350, 300, 150 or 50 mg; suitable values in the ranges being 750, 375, 150, 125 or 50 mg). The ERβ selective agonist is preferably provided in an amount of 0.03 to 250 mg (preferably the lower end of the range being 0.1 mg; preferably the upper end of the range being 10 mg; the most preferred range being 0.3 to 2.5 mg; a suitable value in the range being 0.3, 0.5, 0.75, 1, 1.25, 1.5, 2 or 2.5 mg). For bicalutamide, a preferred range is 25 to 350 mg (preferably the lower end of the range being 50 mg; preferably the upper end of the range being 300, 150 or 50 mg; suitable values in the ranges being 150 or 50 mg). For flutamide, a preferred range is 100 to 1000 mg, and 750 or 375 mg is a preferred value. For chlormadinone acetate a preferred value is 50 mg. For cyproterone acetate a preferred range is 200 to 300 mg. For nilutamide, a preferred range is 50 to 500 mg, and 300 or 150 mg is a preferred value.

In one embodiment, the daily dose comprises 3 times 250 mg of flutamide (eg, 250 mg administered every 8 hours) or 3 times 125 mg of flutamide (eg, 125 mg administered every 8 hours). In another embodiment, the daily dose comprises 3 times 50 mg of bicalutamide (eg, 50 mg administered every 8 hours) or 1 times 150 mg of bicalutamide.

The products and doses of the invention may be in a form suitable for oral use (for example as tablets, capsules, aqueous or oily suspensions, emulsions or dispersible powders or granules), for topical use (for example as creams, ointments, gels, or aqueous or oily solutions or suspensions; for example for use within a transdermal patch), for parenteral administration (for example as a sterile aqueous or oily solution or suspension for intravenous, subcutaneous, intramuscular or intravascular dosing), or as a suppository for rectal dosing. Preferably the products and doses of the invention are in a form suitable for oral use, for example as tablets or capsules.

The products and doses of the invention may be obtained by conventional procedures using conventional pharmaceutically-acceptable diluents or carriers that are well known in the art.

Suitable pharmaceutically-acceptable diluents or carriers for a tablet formulation include, for example, inert diluents such as lactose, sodium carbonate, calcium phosphate or calcium carbonate, granulating and disintegrating agents such as corn starch or alginic acid; binding agents such as gelatin or starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin or olive oil.

It has been observed that the administration of an anti-androgen (bicalutamide) in single agent therapy to humans causes an increase in the amount of testosterone circulating in the blood. Blackledge et al, (Urology, 1996, 47, Suppl. 1A), pp 44-47) discloses an approximate doubling of the basal level of total testosterone. It is believed that such an increase in the level of testosterone occurs when sufficient of the anti-androgen gains access to the CNS and blocks androgen receptors in the hypothalamus. The consequential lack of feedback of androgen causes additional release of LHRH by the hypothalamus which in turn causes release of luteinising hormone (LH) and follicle stimulating hormone (FSH) by the pituitary gland and production of testosterone in the testes. Aromatase enzyme in fat and other tissues converts some of the increased concentration of testosterone to oestradiol, which results in increased concentrations of oestrogen in the blood. Further discussion of this is provided by C Mahler et al, Clinical Pharmacokinetics, 1998, 34(5), pp 405-417. A disadvantageous effect is produced. Namely, the increase in the levels of circulating oestrogen may cause one or more of the side effects of gynaecomastia, breast tenderness, hot flushes, impotence and reduction in libido. A discussion on gynaecomastia can be found in C J Tyrrell, Prostate Cancer and Prostatic Diseases, 1999, 2(4): pp 167-171.

To this end, in one aspect the invention further includes an aromatase inhibitor or an anti-oestrogen with the aim of suppressing increase in the incidence or severity of at least one side effect selected from gynaecomastia, breast tenderness, hot flushes, impotence and reduction in libido. A suitable aromatase inhibitor is anastrozole, known by the AstraZeneca trade name ARIMIDEX™. Anastrozole is known as 2,2′-[5-(1H-1,2,4-triazol-1-ylmethyl)-1,3-phenylene]di(2-methyl-propionitrile), which is disclosed in U.S. re-issue Pat. No. 36,617. A suitable anti-oestrogen is tamoxifen, known by the AstraZeneca trade name NOLVADEX™. Tamoxifen is the trans isomer of 1-(p-beta-dimethylaminoethoxyphenyl)-1,2-diphenylbut-1-ene, which is disclosed in U.S. Pat. No. 4,536,516.

The anti-androgen and anti-oestrogen (eg, tamoxifen) are provided in a ratio respectively of 25 to 1000 mg (or any of the other values specified above for the anti-androgen): 0.5 to 100 (preferably the lower end of the range being 1 or 5; preferably the upper end of the range being 40, 20 or 10; a suitable value in the range being 20). Where an aromatase inhibitor is used, the anti-androgen and aromatase inhibitor (eg, anastrozole) are provided in a ratio respectively of 25 to 1000 (or any of the other values specified above for the anti-androgen): 0.005 to 100 (preferably the lower end of the range being 0.05 or 0.5; preferably the upper end of the range being 50, 10 or 1; the most preferred range being 0.5 to 1; a suitable value in the range being 1).

A suitable daily pharmaceutical dose comprises from 0.5 to 100 mg of tamoxifen or a pharmaceutically acceptable salt or solvate thereof. Preferably, the lower end of the range is 1 or 5 mg; preferably the upper end of the range is 40, 20 or 10 mg; a suitable value in the range being 20 mg. Where an aromatase inhibitor is used, a suitable daily pharmaceutical dose comprises from 0.005 to 100 mg of anastrozole or a pharmaceutically acceptable salt or solvate thereof. Preferably, the lower end of the range is 0.05 or 0.5 mg; preferably the upper end of the range is 50, 10 or 1 mg; the most preferred range is 0.5 to 1 mg; a suitable value in the range being 1 mg.

Experimental Methods

Both in vitro experimental methods and in vivo experimental methods in animals and/or appropriate clinical trials in humans can be used to assess the synergistic activity of the pharmaceutical product of the present invention.

In Vitro Methods

Androgen-dependent or androgen-independent human prostate cancer cell lines can be exposed in vitro to various concentrations of either the anti-androgen or ERβ selective agonist component of the product of the present invention or to various concentrations of a combination of both components. Thereby the extent and duration of the effect of the combination can be determined. For example, human prostate DU145 cells, TSU-PR1 cells, CWR22 cells, PC-3 cells or LNCaP cells can be used. Growth inhibition can be assessed using, for example, a standard soft agar colony-forming assay or, for example, a standard MTT assay. Cellular apoptosis can be assessed using, for example, a standard ELISA assay, for example the Cell Death Detection ELISA Plus Kit available from Boehringer, Mannheim, Germany. Androgen receptor expression can be measured using, for example, Taqman™ PCR or immunohistochemistry. Thereby, it can be shown that, for example, an increased inhibition of cell growth is obtained with a combination of an anti-androgen and an ERβ selective agonist than the maximum obtainable effect of either component of the combination when used alone at concentrations that are not grossly cytotoxic and, for example, the dose response curve for either component can be shifted to show greater potency when the combination is used. It may also be shown, for example, that an ERβ selective agonist is able to regulate androgen receptors.

In Vivo Methods

Tumours derived from prostate cancer tissue or cell lines can be grown in animals such as rats or mice, particularly athymic nude mice or rats. After inoculation or implantation and growth of the tumour cells or tissue, the test animals can be treated with the invention and the size of the tumour before, during and after each treatment schedule can be assessed to provide an indication of the therapeutic effect of the treatment.

For example, a xenograft model can be used involving the implantation and growth of Dunning R-3327 H prostate cancer tissue in adult male inbred Copenhagen rats according to the general procedures disclosed by J T Isaacs et al., Cancer Research 1981, 41 5070-5075 and Cancer Research, 1989, 49 6290-6294 and by D J George et al., Cancer Research, 1999, 59 2395-2401. Test compounds can be suspended in, for example, Tween 80 (registered trademark) by ball-milling, for example for about 16 hours, and dosed orally by gavage. An assessment is made that the oral administration of a combination product of the anti-androgen bicalutamide and the ERβ selective agonist causes substantial reductions in tissue proliferation, for example as measured by conventional Ki67 immunostaining of excised xenograft tissue, and a substantial and sustained reduction in the tumour growth rate.

For example, using a xenograft model involving the implantation and growth of human CWR22 androgen-dependent prostate cancer in male nude mice according to the general procedures disclosed by T G Pretlow et al., Cancer Research 1994, 54 604-6052 and Cancer Research, 1996, 56 3042-3046, an assessment can be made that the oral administration of a combination product of the anti-androgen bicalutamide and ERβ selective agonist causes substantial reductions in tissue proliferation, for example as measured by conventional Ki67 immunostaining of excised xenograft tissue, and a substantial and sustained reduction in the tumour growth rate.

For example, using a xenograft model involving the implantation and growth of human PC-3 or TSU-PR1 prostate cancer in male nude mice, an assessment can be made that the oral administration of a combination product of the anti-androgen bicalutamide and ERβ selective agonist causes a substantial and sustained reduction in the tumour growth rate.

Human Clinical Trial

Patients presenting with prostate cancer will be assessed for disease stage and PSA level will be used as an appropriate tumour marker. Patients with appropriate entry criteria will be allocated to the clinical programme. One group of patients will be dosed orally with the anti-androgen bicalutamide and a second group of patients will be dosed orally with a combination of the anti-androgen bicalutamide and an ERβ selective agonist. Blood samples will be taken periodically and analysed for the level of PSA. Localised prostate tumour growth will be assessed using one or more of digital rectal examination (DRE), computer assisted tomography (CAT) scanning and prostate tissue biopsy sampling. Clinical responses will be defined using conventional criteria. For example, a complete response will indicate that the tumour mass has regressed totally, a partial response will be defined as a 50% or greater reduction in the original tumour volume and stable disease will be defined as a reduction in tumour volume of less than 50% or no increase in tumour volume.

A corresponding trial in patients presenting with BPH can also be conducted. Estrogen Receptor Binding assays to determine specificity:

The ability of a compound to bind to an estrogen receptor can be measured by its ability to compete for binding with the radio-labeled estrogen, [¹²⁵I]-16α-iodo-3,17β-estradiol (NEN, Cat.#NEX-144). The radio-ligand is hereafter referred to as [¹²⁵I]-estradiol.

ER-β (Gen Bank Accession #X99101) or ER-α (Gen Bank Accession #M12674) cDNAs can be cloned into the expression vector pSG5 (Stratagene), transformed into E. coli as described below, and purified using anion-exchange resin columns (Qiagen Cat. #12125). Receptor protein can then be prepared by in vitro transcription and translation of these plasmids using the TNT T7 Quick-Coupled reticulocyte lysate system (Promega Cat. #L1170). Reticulocyte lysate (12.5 ml) is incubated for 90 min at 30° C. with 312.5 μg of ER-α and 625 μg of ER-β plasmids. Programmed lysate is then aliquotted and stored frozen at −80° C.

Test compounds are tested in duplicate at half-log concentrations ranging from 10 pM to 3 μM. The compounds are prepared as 1 mM stocks in DMSO, then diluted in the binding-assay buffer (in mM: 20 HEPES, 150 NaCl, 1 EDTA, 6 monothioglycerol and 10 Na₂MoO_{4,}; 10% wt/vol glycerol, and pH=7.9) to a series of three-fold concentrated, 20 μl aliquots in a 96-well plate. Receptor aliquots are thawed on ice, and appropriately diluted (see below) in binding assay buffer. Diluted receptor (30 μl/each) is added to each well. [¹²⁵I]-estradiol is diluted from the manufacturer's ethanol stock solution to a 900 pM working solution in binding-assay buffer. The final assay volume is 60 μl, consisting of 20 μl of a test compound, 30 μl of programmed reticulocyte lysate, and 10 μl of 900 pM [¹²⁵I]-estradiol. The final concentration of [¹²⁵I]-estradiol is 150 pM. Plates containing the final assay mixture are mixed on a shaker for 2 min and incubated overnight (˜16 h) at 4° C.

Receptor-bound and unbound radioligand is separated by filtration over sephadex columns. Columns (45 μl bed volume) are prepared by adding dry column media (Pharmacia Cat #G-25) to 96-well column templates (Millipore MultiScreen Plates Cat #MAHVN4510). Columns are then saturated with 300 μl of binding-assay buffer and stored at 4° C. Prior to use, stored columns are spun for 10 minutes at 2000 RPM, then washed twice with 200 μl of fresh binding buffer. The binding-assay mixtures (50 μl/each) are then applied to the columns, and an additional elution volume of 35 μl is immediately applied to the column. Receptor-bound radioligand is then eluted from the column by centrifugation for 10 minutes at 2000 RPM. A scintillation cocktail (145 μl) is added to the eluted radioligand/receptor complex, and radio-label is measured by liquid scintillation counting.

Non-specific binding is defined by competition with 150 nM diethylstilbesterol (DES). Binding affinities are expressed as Ki, calculated using the Cheng-Prushoff formula according to IC₅₀ values generated by fitting the relationship of concentration to percent specific binding (SB) with the following equation:

% SB=Maximum−(Maximum−Minimum)/(1+10^{(log IC50-log [compound])})

In this assay, standard estrogen receptor ligands estradiol and DES are detected as high-affinity (K_{i<}1 nM), non-selective ligands of ER-β and ER-α.

The volume of receptor-programmed reticulocyte lysate to be added to the binding assay can be determined independently from two measurements made on each batch of receptor prepared. First, Kᵢs are determined for standard compounds using a series of dilutions of the receptor preparation. Scatchard analysis of ligand binding affinity is performed at the receptor dilutions that produced reported Kᵢs for these compounds and an acceptable signal:noise ratio (˜10). Such experiments indicated a K_{D} for [^{125I]-estradiol of} 0.1-1 nM, and a Bₘₐₓ of 5-30 pmol.

Cell-Based Assay for ER Transcriptional Activity:

ERs are ligand-dependent transcription factors that bind the promoter regions of genes at a consensus DNA sequence called the estrogen responsive element (ERE). The ER agonist or antagonist activity of a drug was determined by measuring the amount of reporter enzyme activity expressed from a plasmid under the control of an estrogen-responsive element when cells transiently transfected with ER and the reporter plasmid were exposed to drug. These experiments were conducted according to the following methods.

Plasmids:

Estrogen Receptors alpha (αER, Gen Bank accession #M12674), and beta (βER, Gen Bank #X99101 were cloned into the expression vector pSG5 (Stratagene). A trimer of the vitellogenin-gene estrogen response element (vitERE) was synthesized as an oligonucleotide and attached to a beta-globin basal promoter in a construct named pERE3gal. This response element and promoter were removed from pERE3gal by digestion with the endonucleases SpeI (filled with Klenow fragment) and HindlIl. This blunt/Hind III fragment was cloned into the β-galactosidase (β-gal) enhancer reporter plasmid (pBGALenh, Stratagene). αER and βER plasmids were purified using the Endo Free Maxi Kit (Qiagen), and the DNA concentration and purity (A260/280 ratio) were determined spectrophotometrically (Pharmacia). Only DNA with A260/280 ratio of 1.8 and a concentration of >1 ug/uL was used for transfections.

Vitellogenin Response Element Sequence:

```
CTAGTCTCGAGAGGTCACTGTGACCTAGATCTAGGTCACTGTGACCT
AGATCTAG
GTCACTGTGACCTAC
                  =SpeI overhang
                  =XhoI site
                  =AflII overhang
                  =ERE consensus
                  =spacer Bgl II
```

Cells:

All Transfections are performed in 293 cells (Human Embryonic Kidney cells ATCC #CRL-1573). Cells are grown in DMEM supplemented with 10% FBS, glutamine, sodium pyruvate and penicilin/streptomycin. Cells are grown to 70% confluency and split 1:4.

Transfection:

1. 293 cells are split the night before onto collagen I-coated 150 mm tissue-culture plates (Biocoat, Becton Dickinson #354551) at a density of 60-70% in DMEM (Mediatech 17-205-CV) 10% charcoal-stripped FBS (biocell #6201-31). Approximately 1×10⁷ cells/plate will yield 70% confluency.

2. The next morning, 1 hour prior to transfection, the media is changed to fresh DMEM 10% FBS stripped and supplements.

3. Transfections are performed using the Profection Kit (Promega #E1200). This kit is based on the calcium-phosphate-mediated transfection technique. Reagents are added in sterile polystyrene tubes in the following order:

Solution A

15 μg αER or βER

45 μg Reporter (pBGALenh or ERE3)

1.5 mL Sterile Water

186 μL CaCl₂

* Mix gently

Solution B

1.5 mL 2X Hank's Buffered Salt Solution

4. Using a vortex set on low, add solution A to solution B dropwise. The resulting solution should become milky in color. It is important to achieve thorough mixing. The solution is allowed to settle for 30 minutes, then vortexed before adding the solution to cells.

5. Add the mixture to 150 mm plates dropwise. Mix well by rocking plates back and forth and side to side gently. After an hour, a very fine precipitate should be seen floating on and above cells under 20× magnification. If this precipitate is not observed, the transfection will not be effective. Incubate the cells for 12 hours.

Receptor Stimulation:

1. The day after transfection, cells are washed 2× with calcium- and magnesium-free Mg free PBS containing 1 mM EGTA (pH 7.6). Cells are trypsinized for 2 min with 3 mL of trypsin-EDTA. Trypsin is neutralized with DMEM 10% FCS. Cells are pelleted at 1000×g for 5 min. The cell pellet is then resuspended in 5 mL DMEM plus 2% phenol-red-free FCS supplemented with glutamine, pyruvate, and Penn/Strep.

2. 50 μl of the resulting cell suspension is plated into each well of 96-well tissue culture dishes (Biocoat B&D #354407) using a multi-channel pipettor. The dishes have been previously loaded with 50 μL of DMSO-solubilized test compounds at twice the test concentration in DMEM. Data reported are either n=4 wells (single poke) and n=2 wells (9-point concentration-response curves).

3. Cells are incubated overnight at 37° C. in the selected compounds.

Reporter Assay:

1. After 24 h, 100 μL of 7% CPRG (Roche 0884308) cocktail is added to each well in 1× Z-buffer, the plate is shaken gently at 37° C. for 3 h. CPRG turns bright red as it is cleaved by β-galactosidase.

2. Absorbance measurments (570 nm) were obtained using a plate reader (Molecular Devices).

3. Data is compiled and analyzed using MS Excel.

10× Z Buffer

| | |
|---|---|
| Sodium Phosphate (dibasic) 1.7 g | 600 mM |
| Sodium Phosphate (monobasic) 0.96 g | 400 mM |
| Potassium Chloride 149 mg | 100 mM |
| Magnesium Sulfate 0.2 mL of 1 molar stock | 100 mM |
| BME 0.78 mL | 500 mM |
| Bring Final Volume to 20 mL with De-Ionized Water | |

7% CPRG COCKTAIL
For 50 mLs:
add 3.5 mL of 50 ml of CPRG
add 3.5 mL of 10× Z Buffer
add 1 mL of 10% SDS
bring to 50 mL with DI water

1. A pharmaceutical product comprising an anti-androgen and an oestrogen receptor β (ERβ) selective agonist, for simultaneous or sequential administration to a patient for therapeutically treating and/or preventing an androgen-stimulated disease in the patient.

2. A pharmaceutical product comprising an anti-androgen, an oestrogen receptor β (ERβ) selective agonist and a chemical castration agent, for simultaneous or sequential administration to a patient for therapeutically treating and/or preventing an androgen-stimulated disease in the patient.

3. The pharmaceutical product of claim 2, wherein the chemical castration agent is selected from goserelin and leuprorelin.

4. The pharmaceutical product of claim 1 or 2, wherein the anti-androgen is selected from flutamide, nilutamide, bicalutamide or a pharmaceutically acceptable salt, enantiomer or solvate thereof, chlormadinone acetate and cyproterone acetate.

5. The pharmaceutical product of claim claim 1 or 2, wherein the ERβ selective agonist is selected from the group consisting of: genistein, diadzen and coumesterol, an oestrogenic analogue thereof, or a pharmaceutically acceptable salt, enantiomer or solvate ERβ selective agonist thereof.

6. The pharmaceutical product of claim 1 or 2, wherein the anti-androgen and the ERβ selective agonist are provided in a weight ratio of 25 to 1000:0.03 to 250 respectively.

7. A daily pharmaceutical dose for administration to a patient for therapeutically treating and/or preventing an androgen-stimulated disease in the patient, the dose comprising an anti-androgen and an ERβ selective agonist, for simultaneous or sequential administration to the patient.

8. The dose of claim 5, comprising from 25 to 1000 mg of the anti-androgen.

9. The dose of claim 5, comprising from 0.03 to 250 mg of the ERβ selective agonist.

10. A pharmaceutical composition comprising the product of claim 1 or 2 and a pharmaceutically acceptable diluent or carrier.

13. The method according to claim 14 or 15, wherein the androgen-stimulated disease is selected from prostate cancer, benign prostate hypertrophy, acne and hirsutism.

14. The method according to claim 14, comprising administering to the patient a pharmaceutical product according to claim 3.

15. A method of therapeutically treating and/or preventing an androgen-stimulated disease in a patient, comprising simultaneously or sequentially administering an anti-androgen and an ERβ selective agonist to the patient.

16. A method of therapeutically treating and/or preventing an androgen-stimulated disease in a patient, comprising simultaneously or sequentially administering an anti-androgen, a chemical castration agent and an ERβ selective agonist to the patient.

17. The pharmaceutical product of claim 1, comprising an aromatase inhibitor or an anti-oestrogen.

18. The dose of claim 6, comprising from 0.03 to 250 mg of the ERβ selective agonist.
